(19) United States
(12) Patent Application Publication
(10) Pub. No.: US 2013/0102661 A1
(43) Pub. Date: Apr. 25, 2013

(54) IRRIGATION SOLUTIONS CONTAINING ASCORBIC ACID OR ITS SALTS AND USE THEREOF

(71) Applicant: Kai Ming Chan, (US)

(72) Inventor: Kai Ming Chan, (US)

(73) Assignee: The Chinese University of Hong Kong, Hong Kong (CN)

(21) Appl. No.: 13/280,539

(22) Filed: Oct. 25, 2011

(51) Int. Cl. A61K 31/375; A61P 17/02; A61P 19/04

(52) U.S. Cl. 514/474

(57) ABSTRACT

An irrigation solution containing about 15-50 mM ascorbic acid and/or a pharmaceutically acceptable salt thereof, and electrolytes, and their uses in a surgery, a surgical manipulation, a medical process or in promoting the healing of a tendon or ligament injury in a subject.

### TECHNICAL FIELD

This application relates to field of the medical sciences and, in particular, relates to irrigation solution for use in medical manipulations.

### BACKGROUND

Currently, musculoskeletal disorders are a leading cause of disability around the world, accounting for 25% of the total cost of illness. The increased recreational sport and elite consideration have drawn more societal concerns on effective treatment for sport injuries, including various forms of ligament and tendon injuries, which have taken up significant health care resources. These injuries have notoriously slow healing rates and compromised mechanical restoration. However, medical research to improve healing outcomes of tendon and ligament injuries has lagged far behind.

### SUMMARY

In an embodiment, there is provided an irrigation solution containing about 15 to about 50 mM ascorbic acid and/or a pharmaceutically acceptable salt thereof, and electrolytes, having osmolarity in the range from about 270 mOsm to about 340 mOsm.

In another embodiment, there is provided a method for treating tendon and/or ligament injury in a subject comprising irrigating the tendon and/or ligament with the irrigation solution disclosed herein.

In another embodiment, there is provided use of ascorbic acid and/or a physiologically acceptable salt thereof in the manufacture of an irrigation solution for promoting the healing in tendon and/or ligament injury of a subject, wherein the concentration of the ascorbic acid and/or a physiologically acceptable salt thereof in the irrigation solution is about 15 to about 50 mM, and the irrigation has an osmolarity in the range from about 270 mOsm to about 340 mOsm.

### BRIEF DESCRIPTION OF THE DRAWINGS

The accompanying drawings, which are incorporated into and form a part of the specification, illustrate one or more embodiments of the present invention and, together with the description, serve to explain the principles of the embodiments disclosed herein. The drawings are only for the principles of illustrating one or more embodiments and are not to be construed as limiting the embodiments. In the drawings:

FIG. 1 is a graph of the elastic moduli of healing tendons using the surgical irrigation solution containing Na vitamin C compared with that of the physiological saline solution in a rat model.

FIG. 2 is a graph of the ultimate tensile strength of healing tendons using the surgical irrigation solution of Na vitamin C compared with that of the physiological saline solution in a rat model.

FIG. 3 is a graph of the static antero-posterior (A-P) knee joint laxity after receiving ACL reconstruction using the surgical irrigation solution containing Na vitamin C compared with that of the physiological saline solution in a rat model.

FIG. 4 is a graph of knee joint stiffness after receiving ACL reconstruction using the surgical irrigation solution containing Na vitamin C compared with that of the physiological saline solution in a rat model.

### DETAILED DESCRIPTION OF THE PREFERRED EMBODIMENTS

### Definitions

As used herein the term “subject” or “patient” is not limited to human beings, but is intended to include all vertebrate animals in addition to human beings. In some embodiments disclosed herein, the terms “patient” and “subject” can be used alternately and refer to an individual undergoing a surgical operation, or suffering from tendon and/or ligament injury.

As used herein, the term “osmolarity” refers to the concentration (osmoles, Osm) of the dissolved substances (solute) per liter of solution that contributes to a solution's osmotic pressure.

As used in this application, the singular form “a”, “an” and “the” include plural references unless the context clearly dictates otherwise. In particular, where the indefinite article is used, the specification is to be understood as contemplating plurality as well as singularity, unless the context requires otherwise.

As used herein, the term “about” or “approximately” when used in conjunction with a number refers to any number within 1 to 5% variant of the referenced number.

Further scope of the applicability of the embodiments will become apparent from the detailed description and drawings provided below. It should be understood, however, that the following detailed description and examples, while indicating embodiments of the invention, are given by way of illustration only, since various changes and modifications within the spirit and scope of the inventions will become apparent to those skilled in the art from the following detailed description.

All publications, patents, patent applications and other references cited in this application are herein incorporated by reference in their entirety as if each individual publication, patent, patent application or other reference were specifically and individually indicated to be incorporated by reference.

In one embodiment, there is provided an irrigation solution containing about 15 to about 50 mM ascorbic acid and/or a pharmaceutically acceptable salt thereof, and electrolytes, having osmolarity in the range from about 270 mOsm to about 340 mOsm.

In another embodiment, there is provided a method for treating tendon or ligament injury in a subject comprising irrigating the tendon or ligament with the irrigation solution disclosed herein.

In still another embodiment, there is provided use of ascorbic acid and/or a pharmaceutically acceptable salt thereof in the manufacture of an irrigation solution for promoting the healing in tendon or ligament injury of a subject, wherein the irrigation solution contains electrolytes in addition to the ascorbic acid and/or a physiologically acceptable salt thereof, and has an osmolarity in the range from about 270 mOsm to 340 mOsm.

In one embodiment, the irrigation solution is useful in a surgery, a surgical manipulation, or a medical process.

In another embodiment, the surgery is selected from the group consisting of an orthopedic surgery, an arthroscopic surgery and an open wound surgery.

In other embodiments, the irrigation solution is a surgical irrigation solution.

In another embodiment, the irrigation solution is for use in promoting the healing in tendon or ligament injury of a subject.

In another embodiment, the osmolarity of the irrigation solution disclosed herein is in the range from about 290 mOsm to about 310 mOsm.

In another embodiment, the irrigation solution is compatible to the physiology of the tissue to be applied to or is isotonic to the tissue.

In a particular embodiment, the irrigation solution has an osmolarity of 298 mOsm.

In one embodiment, the pharmaceutically acceptable salt of ascorbic acid is selected from the group consisting of ascorbate sodium, potassium, calcium salts, and combinations thereof.

In another embodiment, the pharmaceutically acceptable salt of ascorbic acid is sodium ascorbate.

In some embodiments, the electrolytes are selected from the group consisting of sodium chloride, potassium chloride, calcium chloride, magnesium sulfate and combinations thereof.

In embodiments disclosed herein, the desired range of the osmotic pressure can be adjusted by adjusting the electrolyte ion or molecule (non-electrolyte substance) concentration of the irrigation solution. In some embodiments, the concentration of the electrolytes or non-electrolyte substances in the irrigation solution can be determined according to the knowledge of the calculation formulation of “1 mOsm (osmolarity)=1 mmol/L ions of the electrolytes or molecules of the non-electrolyte substances” (Zhang, Q, et al. Chapter H “Sterile and aseptic preparations” in “Pharmaceutics”, 1^{st} ed., Peking University Medical Press, 2005, January, pp 113).

In one embodiment, the electrolytes are sodium chloride, and the concentration of which is 99-133 mM of the irrigation solution.

In another embodiment, the irrigation solution consists of 15-50 mM sodium ascorbate and 99-133 mM sodium chloride aqueous solution.

In one embodiment, the irrigation solution disclosed herein has a pH within physiological range, such as in the range of from about 5.5 to 8.5, or about 5.5 to 7.4, or about 5.5 to 6.7.

In other embodiments, the irrigation solution disclosed herein can maintain the pH within the range described above through a buffer system or not. Where the buffer system is not needed, the electrolytes in the irrigation solution can maintain the pH range. Where the buffer system is needed, the buffer system of the solution includes bicarbonate ion or a zwitterionic compound such as N-2-hydroxyethyl piperazine-N′-ethane sulfonic acid, commonly sold under the trade name HEPES.

In some embodiments, the irrigation solution disclosed herein is a safe, non-toxic and stable solution.

Reference throughout this specification to “one embodiment”, or “an embodiment”, or “another embodiment”, or “some embodiments”, or “other embodiment” means that a particular referent feature, structure, or characteristic described in connection with the embodiment is included in at least one embodiment. Thus, the appearance of the phrases “in one embodiment”, or “in an embodiment”, or “in another embodiment”, or “in some embodiments” in various places throughout this specification are not necessarily all referring to the same embodiment. Furthermore, the particular features, structures, or characteristics may be combined replaced in any suitable manner in one or more embodiments.

The exemplary embodiments will be described with respect to healing of tendon and/or ligament surgeries, illustrated by an acute patellar tendon injury model and an anterior cruciate ligament reconstruction (ACLR) model. It is to be understood that examples merely exemplifies embodiments, but may be applied to other methods and types of surgery with similar benefits.

### EXAMPLES

### Example 1

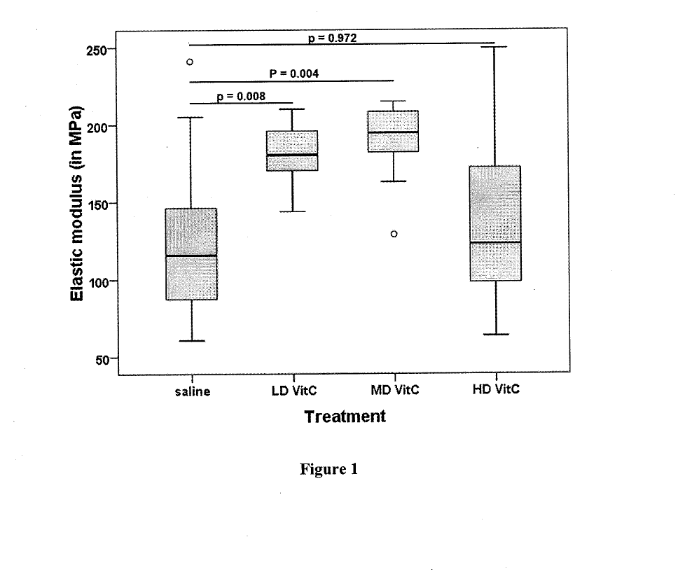

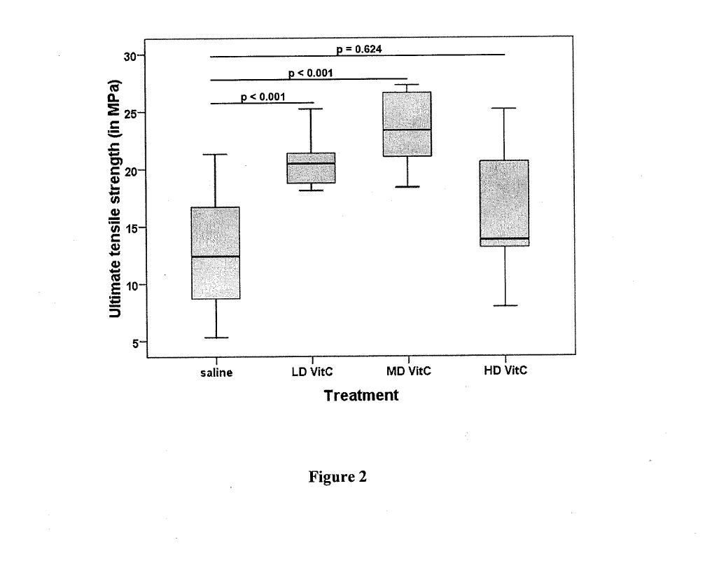

Two-month old male Sprague-dawley rats were used in animal studies to demonstrate the effects of the irrigation solution disclosed herein on the healing of acute tendon injury, using a patellar tendon window injury model. In this example, the irrigation solutions containing 15 mM, 50 mM and 150 mM sodium ascorbate were used in the test groups, while a physiological saline solution was used as control. The effects of the irrigation solution on the mechanical strength of the healing tissue were determined by biomechanical tests after animal sacrifice on day 42. We demonstrated that an initial application of the irrigation solution during surgery is beneficial and promotes healing of the tendon injury. The result of this study is shown in FIG. 1 and FIG. 2.

LD vitC solution contained: 3 mg/ml Na Ascorbate, (or about 15 mM), with 7.8 g/L NaCl

MD vitC solution contained: 10 mg/ml Na Ascorbate, (or about 50 mM), with 5.8 g/L NaCl

HD vit C solution contained 15 mg/ml Na Ascorbate (or about 151 mM) only

As shown in FIG. 1, in a well-established patellar tendon window injury model, SD rats (n=10) received either Physiological saline solution (saline), 15 mM Na Ascorbate (LD VitC), 50 mM Na Ascorbate (MD VitC), 150 mM Na Ascorbate (HD VitC) for irrigation of wound. At 6 week, the elastic moduli of patellar tendons from the LD VitC group (p=0.008) and MD VitC group (p=0.004) were significantly higher than that of the saline group, suggesting that both 15 mM and 50 mM Na Ascorbate are better surgical irrigation solutions than saline for restoring tendon mechanical properties.

As shown in FIG. 2, in the same model, the ultimate tensile strength of LD VitC group (p=0.001) and MD VitC group (p=0.001) were significantly higher than that of the saline group, suggesting that both 15 mM and 50 mM Na Ascorbate are better surgical irrigation solutions than saline for restoring tendon strength.

### Example 2

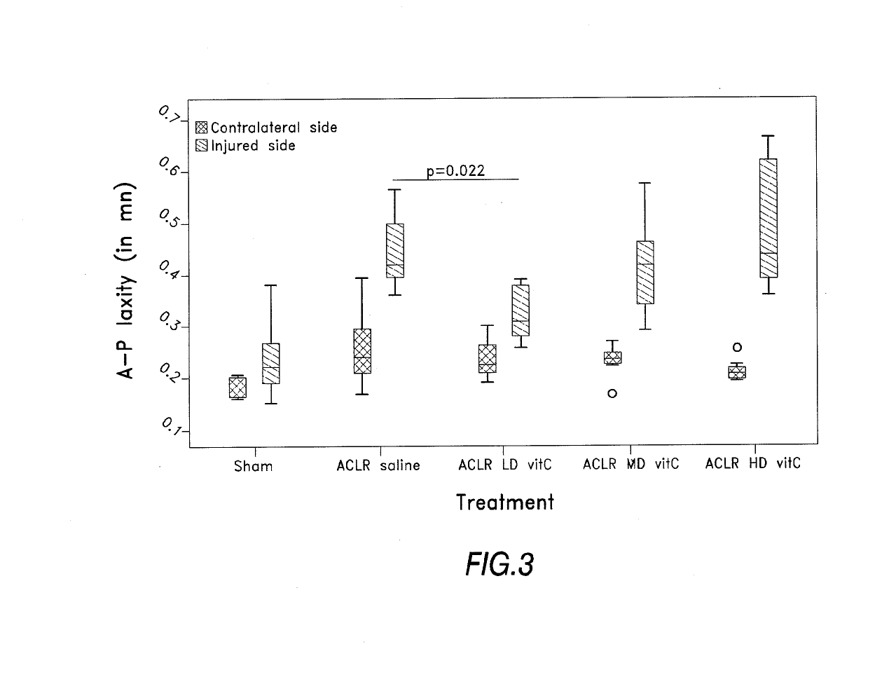

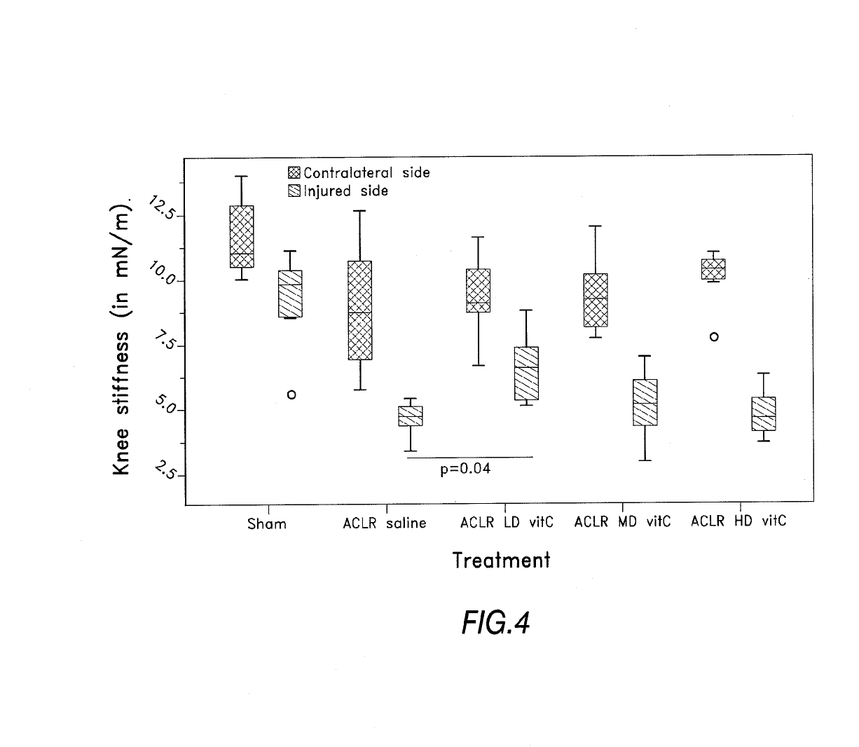

Three-month old male Sprague-dawley rats were used in animal studies to demonstrate the effects of the irrigation solution on healing of ligament injury, using an anterior cruciate ligament (ACL) reconstruction model. The irrigation solutions containing 15 mM, 50 mM and 150 mM sodium ascorbate were used in the test groups, while a physiological saline solution was used as control. The effects of the irrigation solution on the anterior-posterior stability of the knee were demonstrated by biomechanical tests after animal sacrifice on day 42. We demonstrated that an initial application of the irrigation solution during surgery is beneficial and promotes healing of the ACL graft. The result of this study is shown in FIG. 3 and FIG. 4.

As shown in FIG. 3, in a well-established ACL reconstruction model, SD rats (n=8) received either ACL reconstruction or sham operation. Physiological saline solution (ACLR saline), 15 mM Na Ascorbate (ACLR LD vitC), 50 mM Na Ascorbate (ACLR MD vitC) or 150 mM Na Ascorbate (ACLR HD vitC) were used for irrigation of wound during surgery. At week 6, the static A-P laxity of ACLR LD vitC group was significantly lower than that of the ACLR saline group (p=0.022), suggesting that 15 mM Na Ascorbate solution is a better surgical irrigation solution than saline for restoration of A-P knee laxity. Furthermore, the static A-P laxity of ACLR MD vitC group was also lower than that of the ACLR saline group but without statistic significance due to the small sample size. In another experiment with increased sample size, it was demonstrated that 50 mM Na Ascorbate solution is a better surgical irrigation solution than saline for restoration of A-P knee laxity (results not shown).

As shown in FIG. 4, in the same model, knee stiffness from the ACLR LD vitC group was significantly higher than that of ACLR saline group (p=0.04), suggesting that 15 mM Na Ascorbate is a better surgical irrigation solution than saline for restoration of knee properties. Furthermore, knee stiffness from ACLR MD vitC group was also higher than that of the ACLR saline group but without statistic significance due to the small sample size, suggesting that 50 mM Na Ascorbate is a better surgical irrigation solution than saline for restoration of knee properties.

In addition, the animal models treated with the invention did not demonstrate any adverse effects, and scientific reports have confirmed the high safety profile and tolerance of the compounds in humans. Thus the irrigation solution does not impose safety issues during clinical applications.

Natural healing processes of tendon and ligament injuries involve four different stages: inflammation, cell recruitment, tissue repair and remodeling. Previous attempts to promote tendon and ligament healing mainly targeted latter events such as enhancement of cell proliferation and matrix synthesis Experimental results shown in the examples indicate that ascorbic acid and/or pharmaceutically acceptable salts thereof exert positive effects on subsequent mechanical properties of injured tendon and ligaments. These evidences suggest that local application of ascorbic acid and/or pharmaceutically acceptable salts thereof at very early stages of healing or even during a surgical process would have beneficial effects for tendon healing and/or ligament healing.

As ascorbic acid and/or pharmaceutically acceptable salts thereof have a very high safety profile, they gain the merits of high applicability for clinical use. Therefore, the irrigation solutions containing the ascorbic acid and/or pharmaceutically acceptable salts thereof disclosed herein have a broad applicability in the medical field such as for tendon healing and/or ligament healing in surgeries or injuries and will be beneficial to both patients and physicians.

What is claimed is:

1. An irrigation solution comprising
about 15 to about 50 mM ascorbic acid and/or a pharmaceutically acceptable salt thereof, and
electrolytes,
wherein said irrigation solution has an osmolarity in the range from about 270 mOsm to about 340 mOsm.

2. The irrigation solution according to claim 1, wherein the osmolarity of the irrigation solution is in the range from about 290 mOsm to about 310 mOsm.

3. The irrigation solution according to claim 1, wherein the physiologically acceptable salt of ascorbic acid is selected from the group consisting of ascorbate sodium, potassium, calcium salts, and combinations thereof.

4. The irrigation solution according to claim 1, wherein the physiologically acceptable salt of ascorbic acid is sodium ascorbate.

5. The irrigation solution according to claim 1, wherein the electrolytes are selected from the group consisting of sodium chloride, potassium chloride, calcium chloride, magnesium sulfate and combinations thereof.

6. The irrigation solution according to claim 1, wherein the electrolytes are sodium chloride with a concentration of 99-133 mM in the irrigation solution.

7. The irrigation solution according to claim 1, wherein the irrigation solution consists of about 15 to about 50 mM sodium ascorbate and about 99 to about 133 mM sodium chloride aqueous solution.

8. The irrigation solution according to claim 1, wherein the irrigation solution has a pH in the range of from about 5.5 to 8.5, about 5.5 to 7.4, or about 5.5 to 6.7.

9. The irrigation solution according to claim 1, wherein the irrigation solution is used in a surgery, a surgical manipulation, a medical process or in promoting the healing of a tendon or ligament injury in a subject.

10. The irrigation solution according to claim 9, wherein the surgery is selected from the group consisting of an orthopedic surgery, an arthroscopic surgery and an open wound surgery.

11. A method for treating tendon and/or ligament injury in a subject comprising irrigating the tendon and/or ligament an irrigation solution, wherein the irrigation solution contains about 15 to about 50 mM ascorbic acid and/or a pharmaceutically acceptable salt thereof, and electrolytes, and wherein said irrigation solution has osmolarity in the range from about 270 mOsm to about 340 mOsm.

12. The method according to claim 11, wherein the osmolarity of the irrigation solution is in the range from about 290 mOsm to about 310 mOsm.

13. The method according to claim 11, wherein the physiologically acceptable salt of ascorbic acid is selected from the group consisting of ascorbate sodium, potassium, calcium salts, and combinations thereof.

14. The method according to claim 11, wherein the physiologically acceptable salt of ascorbic acid is sodium ascorbate.

15. The method according to claim 11, wherein the electrolytes are selected from the group consisting of sodium chloride, potassium chloride, calcium chloride, magnesium sulfate and combinations thereof.

16. The method according to claim 11, wherein the electrolytes are sodium chloride with a concentration of about 99-133 mM in the irrigation solution.

17. The method according to claim 11, wherein the irrigation solution consists of about 15 to about 50 mM ascorbate sodium and about 99-133 mM sodium chloride aqueous solution.

18. The method according to claim 11, wherein the irrigation solution has a pH in the range of from about 5.5 to 8.5, about 5.5 to 7.4, or about 5.5 to 6.7.

19. The method according to claim 11, wherein the subject is undergoing a surgery, a surgical manipulation, a medical process, or is suffering from tendon and/or ligament injury.

20. The irrigation solution according to claim 19, wherein the surgery is selected from the group consisting of an orthopedic surgery, an arthroscopic surgery and an open wound surgery.
